(19) United States
(12) Patent Application Publication
(10) Pub. No.: US 2023/0133358 A1
(43) Pub. Date: May 4, 2023

(54) SYSTEM AND METHOD FOR MANAGING PATIENT OBSERVATIONS

(71) Applicant: George Jiruska, ambler, PA (US)

(72) Inventor: George Jiruska, ambler, PA (US)

(21) Appl. No.: 17/979,400

(22) Filed: Nov. 2, 2022

Related U.S. Application Data
(60) Provisional application No. 63/274,786, filed on Nov. 2, 2021.

(51) Int. Cl. G16H 40/20; G16H 40/63; G16H 10/60

(52) U.S. Cl. CPC G16H 40/20; G16H 40/63; G16H 10/60

(57) ABSTRACT

A system for managing patient observations is disclosed, including at least one user computing device in operable connection with a user network. An application server is in operable communication with the user network to host an application system managing patient observations. The application system includes a user interface module for providing access to the application system through the user computing device. A patient round module permits the user to input patient data and correspond the patient data to a patient and to handoff patient responsibilities to a second user. The handoff of the patient initiates an offline transfer of patient data between each user. A verification module permits the user to verify their identity during or after the transfer of a patient and while performing patient rounds.

### CROSS-REFERENCE TO RELATED APPLICATIONS

The present application claims priority to U.S. Provisional Application No. 63/274,786 filed Nov. 2, 2021, titled “SYSTEM AND METHOD FOR MANAGING PATIENT OBSERVATIONS,” which is hereby incorporated by reference in its entirety.

### TECHNICAL FIELD

The embodiments generally relate to systems and methods for managing patient observations also known as “safety checks” or “rounds”.

### BACKGROUND

Patient rounds are performed by nurses or behavioral health staff to observe and document patient status, to perform safety checks, and to otherwise evaluate the patient over a period of time that the patient is visiting or interned at a medical care facility. Modernly, electronic rounding systems have been developed and allow caregivers to record medical status information for patients and share the information over a data network and store it in network connected databases. These systems allow caregivers (e.g., doctors, nurses, and others) to input, access, and review medical status information for patients and allow administrative staff to review previously recorded patient information.

### SUMMARY OF THE INVENTION

This summary is provided to introduce a variety of concepts in a simplified form that is disclosed further in the detailed description of the embodiments. This summary is not intended to identify key or essential inventive concepts of the claimed subject matter, nor is it intended for determining the scope of the claimed subject matter.

The embodiments herein relate to a system for managing patient observations. Many embodiments include the use of at least one user computing device coupled to and in operable communication with a user network. An application server is in coupled to and in operable communication with the user network. The application server can host an application system for managing patient observations. The application system can include a user interface module for providing access to the application system through the user computing device. A patient round module permits the user to input patient data that corresponds to individual patients and to handoff or otherwise alert or send patient responsibility information to a second user. The handoff of a patient can trigger or otherwise initiate an offline transfer of patient data between users if adequate internet signal is not present. A verification module requires a user to verify their identity before logging into the system and accepting responsibility for monitoring patients.

Current systems can operate over private networks, the internet, or combinations thereof to transmit information between healthcare professionals. However, many healthcare facilities have poor network or internet connections which causes delays in the transfer of information. Whenever internet/cellular connection is weak or absent, it is difficult or impossible for patient rounding to be performed in a timely manner while maintaining employee accountability. Also, when it is time for a new employee to take over patient rounding responsibilities, it is very challenging to encourage and gain compliance with protocols for a previous rounder logout and a new rounder login to ensure the handoff is timely and smoothly completed. This complication results in long handoff times and the possibility of fraudulent rounding by unauthorized users or by authorized individuals using incorrect credentials.

Further, because existing systems allow a rounder to logout at any time, there is often an urge to stop rounding when a rounder's scheduled time has elapsed or is otherwise complete. This is never acceptable to a hospital, as patient rounding is one of the most important aspects of inpatient behavioral healthcare. Only having the capability to maintain responsibility for a given patient on one computing machine at a time and preventing those machines from logging out until that responsibility is transferred to another rounder puts the accountability firmly in the hands of a single healthcare provider.

The embodiments provided herein allow healthcare professionals to communicate with one another and perform the handoff of patients wirelessly using Bluetooth®, such that communication is not hindered by poor internet connection and can be performed within close physical proximity. The system can also ensure that the healthcare professional responsibly manages their rounds by confirming the completion of their rounds. Further, the system verifies the handoff of a patient between two healthcare professionals.

### BRIEF DESCRIPTION OF THE DRAWINGS

A complete understanding of the present embodiments and the advantages and features thereof will be more readily understood by reference to the following detailed description when considered in conjunction with the accompanying drawings wherein:

FIG. 1 shows a block diagram of a computing system, according to some embodiments;

FIG. 2 shows a block diagram of a computing system and an application program, according to some embodiments;

FIG. 3 shows a block diagram of the application program, according to some embodiments;

and

FIG. 4 illustrates a flow chart of a process for managing patient observations.

### DETAILED DESCRIPTION

The specific details of the single embodiment or variety of embodiments described herein are to the described system and methods of use. Any specific details of the embodiments are used for demonstration purposes only, and no unnecessary limitations or inferences are to be understood thereon.

Before various example embodiments are described in detail, it is noted that the embodiments reside primarily in combinations of components and procedures related to systems. Accordingly, system components have been represented, where appropriate, by conventional symbols in the drawings, showing only those specific details that are pertinent to understanding the embodiments of the present disclosure so as not to obscure the disclosure with details that will be readily apparent to those of ordinary skill in the art having the benefit of the description herein.

In this disclosure, the various embodiments may be systems, methods, and/or computer program products at any possible technical detail level of integration. A computer program product can include, among other things, a computer-readable storage medium having computer-readable program instructions thereon for causing a processor to carry out aspects of the present disclosure.

In general, the embodiments described herein relate to systems for performing safety checks (i.e., rounds) on patients in psychiatric hospitals or other healthcare settings wherein patient rounds are performed. These systems allow healthcare professionals to communicate with one another and to perform patient handoffs wirelessly, using Bluetooth®, such that the communication is not hindered due to poor internet connectivity. These systems may also ensure that healthcare professionals responsibly manage their rounds by confirming they have completed their rounds. Further, these systems verify handoffs of patients between two healthcare professionals.

The system may, at least in part, operate using near-frequency communication (i.e., Bluetooth or similar communication protocol) such that the system may operate offline. The Bluetooth is specifically so patients can be transferred without internet connection. The actual rounding stores on the memory of the device and does not need Bluetooth. This solves a common problem wherein the sharing of patient data is not possible if an online connection cannot be established.

The computing device automatically stores the patient rounding data. Once the network connection is restored and the patient round data is transmitted to the system database, the patient round data is deleted from the computing device storage. In some aspects, the computing device stores patient rounding information during the patient rounding process. In such, the patient rounding information is preserved on the tablet in the event that network connection is lost.

In some embodiments, the system provides the ability to function offline for extended periods of time. In such, the patient data may be stored on the user's computing device to solve internet connectivity problems. In such, the patient data may be stored on the user's computing device and may not be removed until it is successfully uploaded to another database (e.g., once the computing device connects to the internet and the patient data is uploaded to a cloud database or other patient database).

In some embodiments, a biometric authentication is permitted, such that the identity of the healthcare provider is verified. This may be performed by the verification module. In another embodiments, the system may allow for subgroups of healthcare providers (e.g., other physicians, nurses, etc.) the ability to “sign out” a patient and take responsibility for the patient for a given period of time determined by the licensed professional.

In some embodiments, the biometric authentication process is performed at the time of login. The system may also periodically prompt user verification during the logged on period while the user is performing patient rounds.

In some embodiments, the user may be prompted to perform biometric authentication periodically during the patient rounds and/or at the time of patient transfer (i.e., patient handoff).

In some embodiments, the system may only permit one user (i.e., healthcare professional) to be responsible for monitoring a patient profile at a given time. Further, in some embodiments, a patient's profile may only exist (i.e., be viewed and interacted with) by a single user (care provider) at a time.

The system allows a first user to handoff a patient to second user during patient rounds. This process allows the first user to transmit a permission to transfer the patient to the second user. The second user must acknowledge (i.e., by some means accept the patient handoff and therefor the patient round responsibility) the request. This process may occur online or offline depending on network connection status.

During use, a patient rounding profile is created on a tablet which becomes its own “living” document or set of documents. As the patient moves between locations in the hospital, the responsibility to round on that patient may or may not change depending on time of day and location. Whenever the responsibility does change, the rounding profile is transferred to whomever is taking that responsibility on. This can occur without internet/cellular connection if necessary. The timestamp of the transfer may be saved such that the handoff of the patient is confirmed and recorded appropriately.

In some embodiments, the system includes a pedometer module to track user steps throughout the rounding period whether an online network connection or offline status is utilized. Once a patient round is completed, the pedometer is reset to zero such that each round is associated with the user's movement/steps. The pedometer is used as a means of fraud prevention to help ensure that the patient rounds are completed. Information received from the pedometer can be used by management personnel to monitor users, enable reports, and reduce the likelihood of fraudulent or improper rounding of patients.

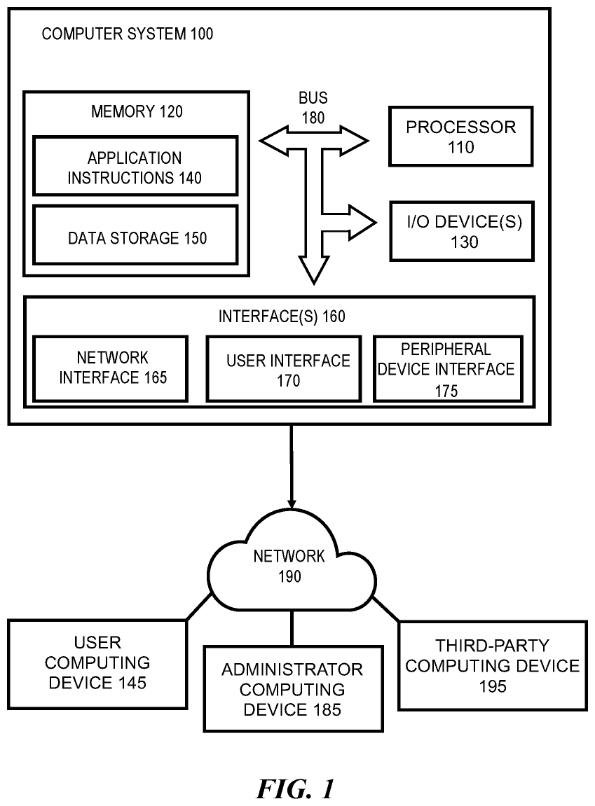

FIG. 1 illustrates an example of a computer system 100 that may be utilized to execute various procedures, including the processes described herein. The computer system 100 comprises a standalone computer or mobile computing device, a mainframe computer system, a workstation, a network computer, a desktop computer, a laptop, or the like. The computing device 100 can be embedded in another device, e.g., a mobile telephone, a personal digital assistant (PDA), a mobile audio or video player, a game console, a Global Positioning System (GPS) receiver, or a portable storage device (e.g., a universal serial bus (USB) flash drive).

In some embodiments, the computer system 100 includes one or more processors 110 coupled to a memory 120 through a system bus 180 that couples various system components, such as an input/output (I/O) devices 130, to the processors 110. The bus 180 may be any of several types of bus structures including a memory bus or memory controller, a peripheral bus, and a local bus using any of a variety of bus architectures. For example, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus, also known as Mezzanine bus.

In some embodiments, the computer system 100 includes one or more input/output (I/O) devices 130, such as video device(s) (e.g., a camera), audio device(s), and display(s) are in operable communication with the computer system 100. In some embodiments, similar I/O devices 130 may be separate from the computer system 100 and may interact with one or more nodes of the computer system 100 through a wired or wireless connection, such as over a network interface.

Processors 110 suitable for the execution of computer readable program instructions include both general and special purpose microprocessors and any one or more processors of any digital computing device. For example, each processor 110 may be a single processing unit or a number of processing units and may include single or multiple computing units or multiple processing cores. The processor(s) 110 can be implemented as one or more microprocessors, microcomputers, microcontrollers, digital signal processors, central processing units, state machines, logic circuitries, and/or any devices that manipulate signals based on operational instructions. For example, the processor(s) 110 may be one or more hardware processors and/or logic circuits of any suitable type specifically programmed or configured to execute the algorithms and processes described herein. The processor(s) 110 can be configured to fetch and execute computer readable program instructions stored in the computer-readable media, which can program the processor(s) 110 to perform the functions described herein.

In this disclosure, the term “processor” can refer to substantially any computing processing unit or device, including single-core processors, single-processors with software multithreading execution capability, multi-core processors, multi-core processors with software multithreading execution capability, multi-core processors with hardware multithread technology, parallel platforms, and parallel platforms with distributed shared memory. Additionally, a processor can refer to an integrated circuit, an application specific integrated circuit (ASIC), a digital signal processor (DSP), a field programmable gate array (FPGA), a programmable logic controller (PLC), a complex programmable logic device (CPLD), a discrete gate or transistor logic, discrete hardware components, or any combination thereof designed to perform the functions described herein. Further, processors can exploit nano-scale architectures, such as molecular and quantum-dot based transistors, switches, and gates, to optimize space usage or enhance performance of user equipment. A processor can also be implemented as a combination of computing processing units.

In some embodiments, the memory 120 includes computer-readable application instructions 150, configured to implement certain embodiments described herein, and a database 150, comprising various data accessible by the application instructions 140. In some embodiments, the application instructions 140 include software elements corresponding to one or more of the various embodiments described herein. For example, application instructions 140 may be implemented in various embodiments using any desired programming language, scripting language, or combination of programming and/or scripting languages (e.g., Android, C, C++, C#, JAVA, JAVASCRIPT, PERL, etc.).

In this disclosure, terms “store,” “storage,” “data store,” data storage,” “database,” and substantially any other information storage component relevant to operation and functionality of a component are utilized to refer to “memory components,” which are entities embodied in a “memory,” or components comprising a memory. Those skilled in the art would appreciate that the memory and/or memory components described herein can be volatile memory, nonvolatile memory, or both volatile and nonvolatile memory. Nonvolatile memory can include, for example, read only memory (ROM), programmable ROM (PROM), electrically programmable ROM (EPROM), electrically erasable ROM (EEPROM), flash memory, or nonvolatile random access memory (RAM) (e.g., ferroelectric RAM (FeRAM). Volatile memory can include, for example, RAM, which can act as external cache memory. The memory and/or memory components of the systems or computer-implemented methods can include the foregoing or other suitable types of memory.

Generally, a computing device will also include, or be operatively coupled to receive data from or transfer data to, or both, one or more mass data storage devices; however, a computing device need not have such devices. The computer readable storage medium (or media) can be a tangible device that can retain and store instructions for use by an instruction execution device. The computer readable storage medium can be, for example, an electronic storage device, a magnetic storage device, an optical storage device, an electromagnetic storage device, a semiconductor storage device, or any suitable combination of the foregoing. A non-exhaustive list of more specific examples of the computer readable storage medium can include: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. In this disclosure, a computer readable storage medium is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

In some embodiments, the steps and actions of the application instructions 140 described herein are embodied directly in hardware, in a software module executed by a processor, or in a combination of the two. A software module may reside in RAM, flash memory, ROM memory, EPROM memory, EEPROM memory, registers, a hard disk, a removable disk, a CD-ROM, or any other form of storage medium known in the art. An exemplary storage medium may be coupled to the processor 110 such that the processor 110 can read information from, and write information to, the storage medium. In the alternative, the storage medium may be integrated into the processor 110. Further, in some embodiments, the processor 110 and the storage medium may reside in an Application Specific Integrated Circuit (ASIC). In the alternative, the processor and the storage medium may reside as discrete components in a computing device. Additionally, in some embodiments, the events or actions of a method or algorithm may reside as one or any combination or set of codes and instructions on a machine-readable medium or computer-readable medium, which may be incorporated into a computer program product.

In some embodiments, the application instructions 140 for carrying out operations of the present disclosure can be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The application instructions 140 can execute entirely on the user's computer, partly on the user's computer, as a stand-alone software package, partly on the user's computer and partly on a remote computer, or entirely on the remote computer or server. In the latter scenario, the remote computer can be connected to the user's computer through any type of network, including a local area network (LAN) or a wide area network (WAN), or the connection can be made to an external computer (for example, through the Internet using an Internet Service Provider). In some embodiments, electronic circuitry including, for example, programmable logic circuitry, field-programmable gate arrays (FPGA), or programmable logic arrays (PLA) can execute the computer readable program instructions by utilizing state information of the computer readable program instructions to personalize the electronic circuitry, in order to perform aspects of the present disclosure.

In some embodiments, the application instructions 140 can be downloaded to a computing/processing device from a computer readable storage medium, or to an external computer or external storage device via a network 190. A network adapter card or network interface in each computing/processing device receives computer readable program instructions from the network and forwards the computer readable application instructions 140 for storage in a computer readable storage medium within the respective computing/processing device.

In some embodiments, the computer system 100 includes one or more interfaces 160 that allow the computer system 100 to interact with other systems, devices, or computing environments. In some embodiments, the computer system 100 comprises a network interface 165 to communicate with a network 190. In some embodiments, the network interface 165 is configured to allow data to be exchanged between the computer system 100 and other devices attached to the network 190, such as other computer systems, or between nodes of the computer system 100. In various embodiments, the network interface 165 may support communication via wired or wireless general data networks, such as any suitable type of Ethernet network, for example, via telecommunications/telephony networks such as analog voice networks or digital fiber communications networks, via storage area networks such as Fiber Channel SANs, or via any other suitable type of network and/or protocol. Other interfaces include the user interface 170 and the peripheral device interface 175.

In some embodiments, the network 190 corresponds to a local area network (LAN), wide area network (WAN), the Internet, a direct peer-to-peer network (e.g., device to device Wi-Fi, Bluetooth, etc.), and/or an indirect peer-to-peer network (e.g., devices communicating through a server, router, or other network device). The network 190 can comprise copper transmission cables, optical transmission fibers, wireless transmission, routers, firewalls, switches, gateway computers and/or edge servers. The network 190 can represent a single network or multiple networks. In some embodiments, the network 190 used by the various devices of the computer system 100 is selected based on the proximity of the devices to one another or some other factor. For example, when a first user device and second user device are near each other (e.g., within a threshold distance, within direct communication range, etc.), the first user device may exchange data using a direct peer-to-peer network. But when the first user device and the second user device are not near each other, the first user device and the second user device may exchange data using a peer-to-peer network (e.g., the Internet). The Internet refers to the specific collection of networks and routers communicating using an Internet Protocol (“IP”) including higher level protocols, such as Transmission Control Protocol/Internet Protocol (“TCP/IP”) or the Uniform Datagram Packet/Internet Protocol (“UDP/IP”).

Any connection between the components of the system may be associated with a computer-readable medium. For example, if software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. As used herein, the terms “disk” and “disc” include compact disc (CD), laser disc, optical disc, digital versatile disc (DVD), floppy disk, and Blu-ray disc; in which “disks” usually reproduce data magnetically, and “discs” usually reproduce data optically with lasers. Combinations of the above should also be included within the scope of computer-readable media. In some embodiments, the computer-readable media includes volatile and nonvolatile memory and/or removable and non-removable media implemented in any type of technology for storage of information, such as computer-readable instructions, data structures, program modules, or other data. Such computer-readable media may include RAM, ROM, EEPROM, flash memory or other memory technology, optical storage, solid state storage, magnetic tape, magnetic disk storage, RAID storage systems, storage arrays, network attached storage, storage area networks, cloud storage, or any other medium that can be used to store the desired information and that can be accessed by a computing device. Depending on the configuration of the computing device, the computer-readable media may be a type of computer-readable storage media and/or a tangible non-transitory media to the extent that when mentioned, non-transitory computer-readable media exclude media such as energy, carrier signals, electromagnetic waves, and signals per se.

In some embodiments, the system is world-wide-web (www) based, and the network server is a web server delivering HTML, XML, etc., web pages to the computing devices. In other embodiments, a client-server architecture may be implemented, in which a network server executes enterprise and custom software, exchanging data with custom client applications running on the computing device.

In some embodiments, the system can also be implemented in cloud computing environments. In this context, “cloud computing” refers to a model for enabling ubiquitous, convenient, on-demand network access to a shared pool of configurable computing resources (e.g., networks, servers, storage, applications, and services) that can be rapidly provisioned via virtualization and released with minimal management effort or service provider interaction, and then scaled accordingly. A cloud model can be composed of various characteristics (e.g., on-demand self-service, broad network access, resource pooling, rapid elasticity, measured service, etc.), service models (e.g., Software as a Service (“SaaS”), Platform as a Service (“PaaS”), Infrastructure as a Service (“IaaS”), and deployment models (e.g., private cloud, community cloud, public cloud, hybrid cloud, etc.).

As used herein, the term “add-on” (or “plug-in”) refers to computing instructions configured to extend the functionality of a computer program, where the add-on is developed specifically for the computer program. The term “add-on data” refers to data included with, generated by, or organized by an add-on. Computer programs can include computing instructions, or an application programming interface (API) configured for communication between the computer program and an add-on. For example, a computer program can be configured to look in a specific directory for add-ons developed for the specific computer program. To add an add-on to a computer program, for example, a user can download the add-on from a website and install the add-on in an appropriate directory on the user's computer.

In some embodiments, the computer system 100 may include a user computing device 145, an administrator computing device 185 and a third-party computing device 195 each in communication via the network 190. The user computing device 145 may be utilized a user (e.g., a healthcare provider) to interact with the various functionalities of the system including to perform patient rounds, handoff patient rounding responsibility, perform biometric verification tasks, and other associated tasks and functionalities of the system. The administrator computing device 185 is utilized by an administrative user to moderate content and to perform other administrative functions. The third-party computing device 195 may be utilized by third parties to receive communications from the user computing device, transmit communications to the user via the network, and otherwise interact with the various functionalities of the system.

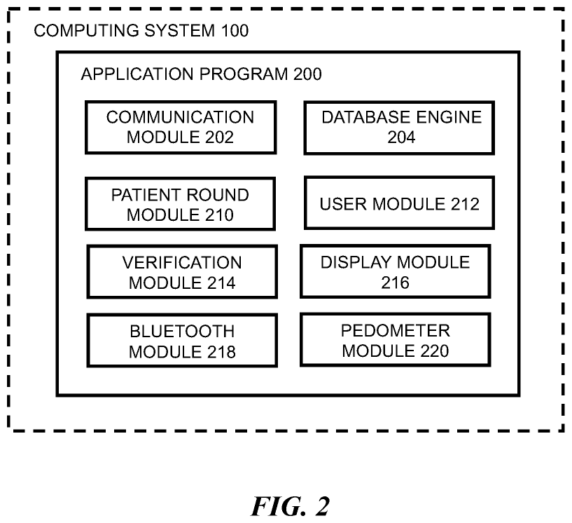

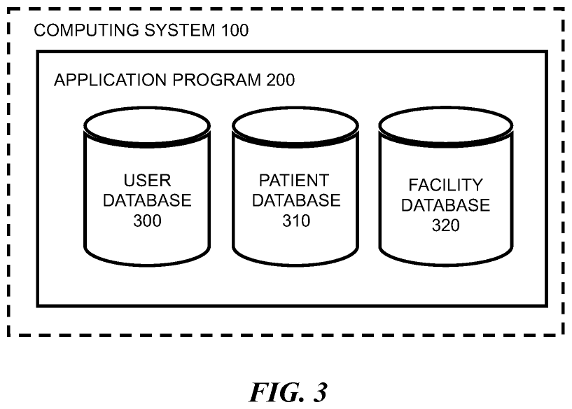

FIGS. 2 and 3 illustrate an example computer architecture for the application program 200 operated via the computing system 100. The computer system 100 comprises several modules and engines configured to execute the functionalities of the application program 200, and a database engine 204 configured to facilitate how data is stored and managed in one or more databases. In particular, FIG. 2 is a block diagram showing the modules and engines needed to perform specific tasks within the application program 200, and FIG. 3 is a block diagram showing the various databases utilized by the various modules.

Referring to FIG. 2, the computing system 100 operating the application program 200 comprises one or more modules having the necessary routines and data structures for performing specific tasks, and one or more engines configured to determine how the platform manages and manipulates data. In some embodiments, the application program 200 comprises one or more of a communication module 202, a database engine 204, a patient round module 210, a user module 212, a verification module 214, a display module 216, a Bluetooth module 218, and a pedometer module 220.

In some embodiments, the communication module 202 is configured for receiving, processing, and transmitting a user command and/or one or more data streams. In such embodiments, the communication module 202 performs communication functions between various devices, including the user computing device 145, the administrator computing device 185, and a third-party computing device 195. In some embodiments, the communication module 202 is configured to allow one or more users of the system, including a third-party, to communicate with one another. In some embodiments, the communications module 202 is configured to maintain one or more communication sessions with one or more servers, the administrative computing device 185, and/or one or more third-party computing device(s) 195.

In some embodiments, the communication module 202 is in operable communication with the patient round module 210 to allow for patient data to be transmitted between user computing devices. The communication module 202 may permit the transmission of patient information during a handoff procedure wherein a first user (i.e., a first caregiver having responsibility of the patient during a round) to a second user (who is assuming responsibility of the patient following the handoff).

In some embodiments, a database engine 204 is configured to facilitate the storage, management, and retrieval of data to and from one or more storage mediums, such as the one or more internal databases described herein. In some embodiments, the database engine 204 is coupled to an external storage system. In some embodiments, the database engine 204 is configured to apply changes to one or more databases. In some embodiments, the database engine 204 comprises a search engine component for searching through thousands of data sources stored in different locations.

In some embodiments, the patient round module 210 is in operable communication with the computing device to allow the user to perform the various tasks of performing patient rounds. For example, the user may input patient information, confirm the completion of each patient round they have performed, as well as initiate a handoff of patient responsibilities to another user.

In some embodiments, the user module 212 facilitates the creation of a user account for the application system. The user module 212 may allow the user to create a user profile which includes user information, user-associated patient information, rounding responsibilities, etc.

In some embodiments, the verification module 214 is in operable communication with the computing device to allow the user to verify their identity prior to or during a patient interaction, when handing off patient responsibility or receiving responsibility for a patient.

In some embodiments, the display module 216 is configured to display one or more graphic user interfaces, including, e.g., one or more user interfaces, one or more consumer interfaces, one or more video presenter interfaces, etc. In some embodiments, the display module 216 is configured to temporarily generate and display various pieces of information in response to one or more commands or operations. The various pieces of information or data generated and displayed may be transiently generated and displayed, and the displayed content in the display module 216 may be refreshed and replaced with different content upon the receipt of different commands or operations in some embodiments. In such embodiments, the various pieces of information generated and displayed in a display module 216 may not be persistently stored.

In some embodiments, the Bluetooth module 218 (also referred to herein as the Bluetooth Module) permits the transmission of patient information between the first user and the second user. In such, the transfer of information may be accomplished in online and/or offline environments. The NFC module 218 and verification module 214 may ensure the first user and second user are within a predetermined range (e.g., 10-feet) of one another during the patient handoff procedure as a component of the identity verification process.

In some embodiments, the pedometer module 220 is in operable communication with the pedometer operated by the computing device. The pedometer module 220 tracks user steps throughout the rounding period whether an online network connection or offline status is utilized. In one example, once a patient round is completed, the pedometer is reset to zero such that each round is associated with the user's movement/steps. The pedometer is used as a means of fraud prevention to help ensure that the patient rounds are completed. Information received from the pedometer can be used by management personnel to monitor users, enable reports, and reduce the likelihood of fraudulent or improper rounding of patients.

FIG. 3 illustrates the computing system 100 in operable communication with the application program 200 having a plurality of databases in communication thereto. A user database 300 is operable to store user information such as user preferences, user profile information, historical usage data, historical content, communications information, biometric information, user credentials, etc. The patient database 310 stores patient information which has been provided to the system by users. The facility database 320 stores facility information and may be in communication with the patient database 310 to send and receive patient information.

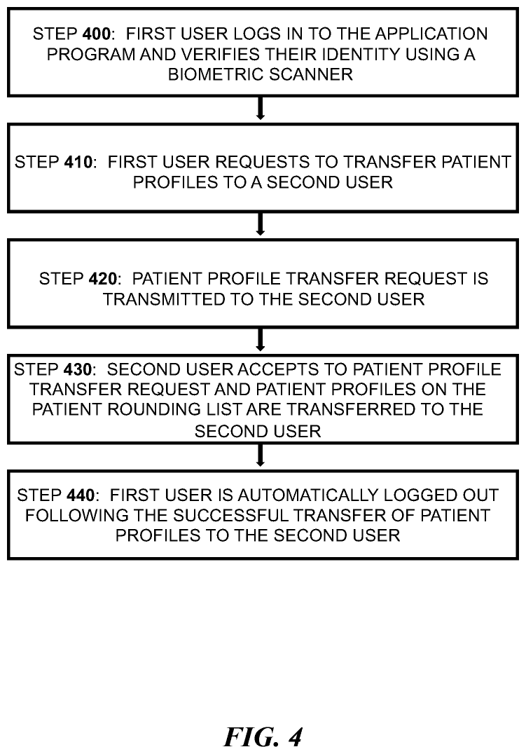

FIG. 4 illustrates a flowchart of the patient rounding process and user verification process. The process allows for the secure and verifiable transfer of patients to ensure the patient rounding is completed in a proper manner. In step 400, the first user logs into the application program using a biometric scanner in communication with the computing device. The biometric scanner may be in communication with the user module and/or verification module to verify the identity of the first user. In step 410, the first user requests to transfer their patient profiles including in the patient rounding list to a second user resulting in the transferring of the request to the second user in step 420. In step 430, the second user accepts the request for the patient profiles to be transferred using the patient rounding module. In step 440, the first user is automatically logged out of the system once each of the patient profiles included in the patient rounding list has been successfully transferred to the second user. Once the patient profiles have been transferred to the second user, the process may start over once a new user (care provider) begins their patient rounds.

In some embodiments, the user is automatically logged out of the system once the final patient has been rounded and is therefore removed from their list. In such, the automatic log out occurs once the patient rounds are completed. I another example, the user is automatically logged out of the system once the final patient (or multiple patients) have been transferred to the second user.

In some embodiments, the transfer of patients, as described in FIG. 4 may be accomplished in an online or offline status.

In some embodiments, the verification module 214 ensures that the user is authentic during the login process and that they maintain accountability for the patients on their device until the appropriate time to handoff this responsibility. This is accomplished through biometric authentication. For example, a first user has 10 patients who they are responsible to round on. The verification module 214 may not deactivate, logoff, or otherwise disassociate from the system until all 10 patients have been handed off to a verified second user using the handoff procedure described in FIG. 4.

In this disclosure, the various embodiments are described with reference to the flowchart illustrations and/or block diagrams of methods, apparatus (systems), and computer program products. Those skilled in the art would understand that each block of the flowchart illustrations and/or block diagrams, and combinations of blocks in the flowchart illustrations and/or block diagrams, can be implemented by computer readable program instructions. The computer readable program instructions can be provided to a processor of a general purpose computer, special purpose computer, or other programmable data processing apparatus to produce a machine, such that the instructions, which execute via the processor of the computer or other programmable data processing apparatus, create means for implementing the functions or acts specified in the flowchart and/or block diagram block or blocks. The computer readable program instructions can be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks. The computer readable program instructions can be loaded onto a computer, other programmable data processing apparatus, or other device to cause a series of operational acts to be performed on the computer, other programmable apparatus, or other device to produce a computer implemented process, such that the instructions that execute on the computer, other programmable apparatus, or other device implement the functions or acts specified in the flowchart and/or block diagram block or blocks.

In this disclosure, the block diagrams in the Figures illustrate the architecture, functionality, and operation of possible implementations of systems, methods, and computer program products according to the various embodiments. Each block in the flowchart or block diagrams can represent a module, segment, or portion of instructions, which comprises one or more executable instructions for implementing the specified logical function(s). In some embodiments, the functions noted in the blocks can occur out of the order noted in the Figures. For example, two blocks shown in succession can, in fact, be executed concurrently or substantially concurrently, or the blocks can sometimes be executed in the reverse order, depending upon the functionality involved. In some embodiments, each block of the block diagrams and/or flowchart illustration, and combinations of blocks in the block diagrams and/or flowchart illustration, can be implemented by a special purpose hardware-based system that performs the specified functions or acts or carry out combinations of special purpose hardware and computer instructions.

In this disclosure, the subject matter has been described in the general context of computer-executable instructions of a computer program product running on a computer or computers, and those skilled in the art would recognize that this disclosure can be implemented in combination with other program modules. Generally, program modules include routines, programs, components, data structures, etc. that perform particular tasks and/or implement particular abstract data types. Those skilled in the art would appreciate that the computer-implemented methods disclosed herein can be practiced with other computer system configurations, including single-processor or multiprocessor computer systems, mini-computing devices, mainframe computers, as well as computers, hand-held computing devices (e.g., PDA, phone), microprocessor-based or programmable consumer or industrial electronics, and the like. The illustrated embodiments can be practiced in distributed computing environments where tasks are performed by remote processing devices that are linked through a communications network. Some embodiments of this disclosure can be practiced on a stand-alone computer. In a distributed computing environment, program modules can be located in both local and remote memory storage devices.

In this disclosure, the terms “component,” “system,” “platform,” “interface,” and the like, can refer to and/or include a computer-related entity or an entity related to an operational machine with one or more specific functionalities. The disclosed entities can be hardware, a combination of hardware and software, software, or software in execution. For example, a component can be a process running on a processor, a processor, an object, an executable, a thread of execution, a program, and/or a computer. By way of illustration, both an application running on a server and the server can be a component. One or more components can reside within a process and/or thread of execution and a component can be localized on one computer and/or distributed between two or more computers. In another example, respective components can execute from various computer readable media having various data structures stored thereon. The components can communicate via local and/or remote processes such as in accordance with a signal having one or more data packets (e.g., data from one component interacting with another component in a local system, distributed system, and/or across a network such as the Internet with other systems via the signal). As another example, a component can be an apparatus with specific functionality provided by mechanical parts operated by electric or electronic circuitry, which is operated by a software or firmware application executed by a processor. In such a case, the processor can be internal or external to the apparatus and can execute at least a part of the software or firmware application. As another example, a component can be an apparatus that provides specific functionality through electronic components without mechanical parts, wherein the electronic components can include a processor or other means to execute software or firmware that confers at least in part the functionality of the electronic components. In some embodiments, a component can emulate an electronic component via a virtual machine, e.g., within a cloud computing system.

The phrase “application” as is used herein means software other than the operating system, such as Word processors, database managers, Internet browsers and the like. Each application generally has its own user interface, which allows a user to interact with a particular program. The user interface for most operating systems and applications is a graphical user interface (GUI), which uses graphical screen elements, such as windows (which are used to separate the screen into distinct work areas), icons (which are small images that represent computer resources, such as files), pull-down menus (which give a user a list of options), scroll bars (which allow a user to move up and down a window) and buttons (which can be “pushed” with a click of a mouse). A wide variety of applications is known to those in the art.

The phrases “Application Program Interface” and API as are used herein mean a set of commands, functions and/or protocols that computer programmers can use when building software for a specific operating system. The API allows programmers to use predefined functions to interact with an operating system, instead of writing them from scratch. Common computer operating systems, including Windows, Unix, and the Mac OS, usually provide an API for programmers. An API is also used by hardware devices that run software programs. The API generally makes a programmer's job easier, and it also benefits the end user since it generally ensures that all programs using the same API will have a similar user interface.

The phrase “central processing unit” as is used herein means a computer hardware component that executes individual commands of a computer software program. It reads program instructions from a main or secondary memory, and then executes the instructions one at a time until the program ends. During execution, the program may display information to an output device such as a monitor.

The term “execute” as is used herein in connection with a computer, console, server system or the like means to run, use, operate or carry out an instruction, code, software, program and/or the like.

In this disclosure, the descriptions of the various embodiments have been presented for purposes of illustration and are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein. Thus, the appended claims should be construed broadly, to include other variants and embodiments, which may be made by those skilled in the art.

What is claimed is:

1. A system for managing patient observations, the system comprising:
at least one user computing device in operable connection with a user network;
an application server in operable communication with the user network, the application server configured to host an application system managing patient observations, the application system having a user interface module for providing access to the application system through the user computing device;
a patient round module to permit the user to input patient data and correspond the patient data to a patient and to handoff patient responsibilities to a second user, wherein the handoff of the patient initiates an offline transfer of patient data between each user;
a verification module to permit the user to verify their identity throughout a rounding process or during a login event to authenticate the user.

2. The system of claim 1, wherein the verification module is in communication with a biometric scanner to biometrically scan the user to verify their identity.

3. The system of claim 1, wherein the offline transfer of patient data is performed using Bluetooth.

4. The system of claim 1, wherein the verification module verifies a status of the patient round prior to the patient handoff to the second user.

5. The system of claim 1, wherein a communication module permits the transmission of patient information between the first user and the second user.

6. The system of claim 1, wherein the verification module retains active communication with the patient round module to maintain the user's online or offline status until each patient on the patient round list has been transferred to the second user.

7. The system of claim 6, wherein the handoff of a patient on the patient round list removes the patient from the user's patient round list and places the patient on the second user's patient round list.

8. The system of claim 1, wherein the communication module permits the transmission of patient information to a patient database accessible by each user.

9. The system of claim 8, wherein the patient database permits the storage of patient information input by the user during a patient interaction.

10. The system of claim 1, further comprising a facility database to permit the storage and transmission of facility information.

11. The system of claim 1, further comprising a pedometer module to monitor the user's movement during the patient rounding process.

12. A method for managing patient observations and performing a patient handoff procedure, the method comprising the steps of:
logging in to an application program using a biometric scanner in operable communication with a computing device;
requesting, via a first user, a transfer of one or more patient profiles included in a patient rounding list;
transferring the request to a second user;
accepting, via the second user, the request;
transferring, via a patient rounding module, the one or more patient profiles included in the patient rounding list;
logging out the first user automatically following the transfer of each of the one or more patient profiles included in the patient rounding list,
wherein the transferring of the one or more patient profiles occurs in an online or offline status.

13. The method of claim 12, wherein the biometric scanner is capable of performing facial recognition to verify the user.

14. The method of claim 12, wherein the verification module retains active communication with the patient round module to maintain the user's online status until each patient has been transferred to the second user.

15. The method of claim 13, wherein the handoff of a patient on the patient round list removes the patient from the user's patient round list and places the patient on the second user's patient round list, and wherein the patient profile is only available to a single user at a time.

16. The method of claim 12, wherein the verification module is in communication with a biometric scanner to biometrically scan the user to verify their identity, wherein the verification module permits the ability to biometrically verify the user at the time of login and/or during a patient rounding period.

17. The method of claim 12, wherein the offline transfer of patient data is performed using Bluetooth.

18. The method of claim 12, wherein the verification module confirms the user is up to date on a patient's rounds before responsibility for the patient can be transferred to another user.

19. The method of claim 12, wherein a communication module permits the transmission of patient information between the user and the second user.

20. The method of claim 12, wherein the communication module permits the transmission of patient information to a patient database accessible by the user.

21. The method of claim 20, wherein the patient database permits the storage of patient information input by the user during a patient interaction.

22. The method of claim 12, further comprising a facility database to permit the storage and transmission of facility information.
